(19) United States
(12) Patent Application Publication
(10) Pub. No.: US 2022/0362028 A1
(43) Pub. Date: Nov. 17, 2022

(54) TIBIAL COMPONENT

(71) Applicant: KYOCERA CORPORATION, Kyoto-shi, Kyoto (JP)

(72) Inventors: Kazutaka SUGIMOTO, Hikone-shi, Shiga (JP); Tomoyuki MIZUGUCHI, Austin, TX (US)

(21) Appl. No.: 17/789,052

(22) PCT Filed: Dec. 14, 2020
(86) PCT No.: PCT/JP2020/046480

§ 371 Date: Jun. 24, 2022

(30) Foreign Application Priority Data
Dec. 26, 2019 (JP) 2019-237296

(51) Int. Cl. A61F 2/38

(52) U.S. Cl. CPC A61F 2/389; A61F 2002/30736

(57) ABSTRACT

In a tibial component, an engagement mechanism configured to engage a tibial block and a tibial tray includes a recessed portion that is disposed in a lower surface, is non-penetrating, and includes an overhanging portion protruding inward more on an opening side than on a bottom portion side in at least a part of an inner wall; and a projecting portion that protrudes from the tibial block to the lower surface side, and includes a protruding part configured to engage with the overhanging portion.

### TECHNICAL FIELD

The present invention relates to a tibial component of an artificial knee joint.

### BACKGROUND ART

A tibial component for forming an artificial knee joint is formed of a tibial plate in contact with a femoral component, a tibial tray including an upper surface for mounting the tibial plate, and the like.

Of the tibial component for a revision operation, an augment member such as a tibial block or a tibial wedge is mounted on a lower surface of the tibial tray, i.e., a surface opposite to the upper surface described above.

A known tibial tray has a through hole extending from the lower surface to the upper surface in order to mount the augment member.

### SUMMARY

### Technical Problem

With the through hole formed in the tibial tray, when a contact surface of the tibial plate and the upper surface of the tibial tray slightly slide against each other, shaving (also referred to as back side wear) of the contact surface of the tibial plate by an edge of the through hole occurs.

A known tibial plate is also known to have a configuration for mounting an augment member without forming the through hole.

One aspect of the present invention realizes a tibial component in which an augment member can be mounted by a simple procedure without requiring a through hole extending from a lower surface to an upper surface of a tibial tray.

### Solution to Problem

<1> A tibial component according to one aspect of the present invention is a tibial component constituting an artificial knee joint, and includes: a tray including a main surface; an augment member disposed on the main surface of the tray; and an engagement mechanism configured to engage the augment member and the tray, wherein the engagement mechanism includes a recessed portion that is disposed in the main surface, is non-penetrating, and includes an overhanging portion protruding inward more on an opening side than on a bottom portion side in at least a part of an inner wall, and a projecting portion that protrudes from the augment member to the main surface side, and includes a protruding part configured to engage with the overhanging portion.

### Advantageous Effects of Invention

According to one aspect of the present invention, a tibial component in which an augment member can be mounted by a simple procedure without requiring a through hole extending from a lower surface to an upper surface of a tibial tray is realized.

### BRIEF DESCRIPTION OF DRAWINGS

FIG. 1 is a schematic view illustrating a configuration example of an artificial knee joint according to one aspect of the present invention.

FIG. 2 is a perspective view illustrating a tibial component.

FIG. 3 is a plan view of a tibial tray viewed from below.

FIG. 4 is a perspective view illustrating a tibial block except for a protruding part.

FIG. 5 is a perspective view illustrating the protruding part of the tibial block.

FIG. 6 is a cross-sectional view of the tibial block including a projecting portion taken along line A-A in FIG. 4.

FIG. 7 is a plan view when the tibial component in which the tibial block and the tibial tray engage with each other is viewed from below.

FIG. 8 is a schematic view for describing one example of an engagement mechanism configured to engage the tibial tray and the tibial block.

FIG. 9 is a schematic view for describing one example of the engagement mechanism configured to engage the tibial tray and the tibial block.

FIG. 10 is a schematic view illustrating one example of a fixing mechanism for fixing the tibial block to the tibial tray.

FIG. 11 is a plan view of a tibial tray viewed from below.

FIG. 12 is a perspective view illustrating a tibial block.

FIG. 13 is a cross-sectional view of the tibial block taken along line B-B in FIG. 16.

FIG. 14 is a schematic view for describing one example of an engagement mechanism for engaging the tibial tray and the tibial block.

FIG. 15 is a schematic view for describing one example of the engagement mechanism configured to engage the tibial tray and the tibial block.

FIG. 16 is a schematic view for describing one example of a fixing mechanism for fixing the tibial block to the tibial tray.

FIG. 17 is a perspective view when the tibial component in which the tibial block is fixed to the tibial tray by using a positioning pin is viewed from below.

FIG. 18 is a schematic view for describing the fixing mechanism for fixing the tibial tray and the tibial block.

FIG. 19 is a schematic view for describing an engagement mechanism for engaging a tibial tray and a tibial block, and a fixing mechanism for fixing the tibial block to the tibial tray.

### DESCRIPTION OF EMBODIMENTS

### First Embodiment

Hereinafter, one embodiment of the present invention will be described in detail.

A knee is a joint formed of three bones of a femur F, a tibia T, and a patella. An artificial knee joint replacement operation can be applied to a patient with a highly impaired knee joint in order to reacquire the original function of the knee joint of the patient again. In the artificial knee joint replacement operation, a bone and a cartilage of a damaged knee joint are removed, and the removed portion is replaced with an artificial replacement (implant) formed of metal or plastic having biocompatibility. The artificial replacement is called an “artificial knee joint”.

### Configuration of Artificial Knee Joint 100

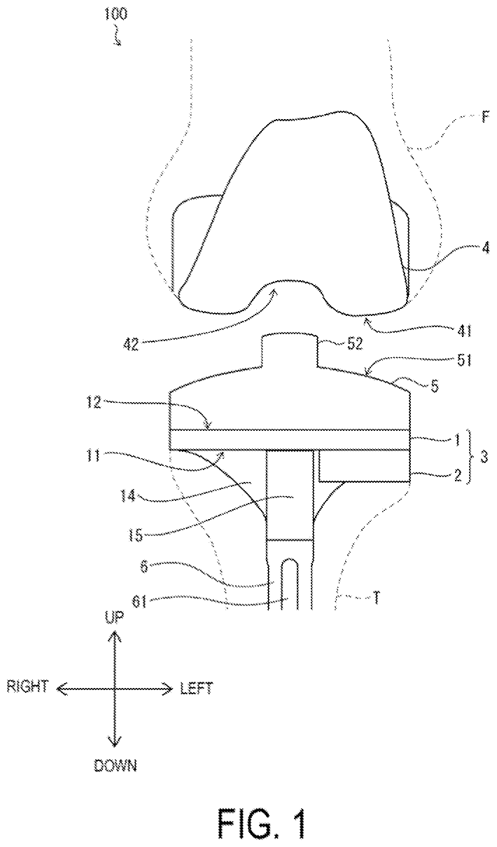

First, a configuration of an artificial knee joint 100 will be described with reference to FIG. 1. FIG. 1 is a schematic view illustrating a configuration example of the artificial knee joint 100 according to one aspect of the present invention. Note that FIG. 1 illustrates, by a dotted line, a contour image of the femur F and the tibia T when the artificial knee joint 100 is disposed in a knee joint of a patient. FIG. 1 illustrates the knee joint when a knee of the patient is viewed from the front. Note that, in FIGS. 1 to 19, a side of a waist of the patient indicates “up”, a side of feet of the patient indicates “down”, a side of a right leg of the patient indicates “right”, and a side of a left leg of the patient indicates “left”.

### Femoral Component 4

A femoral component 4 is used for repair or replacement of the femur F of the patient. The femoral component 4 is designed so as to form a joint with a tibial component 3 or a tibial plate 5. Note that the femoral component 4 may be designed so as to form a joint with a patellar component (not illustrated) as needed. The femoral component 4 is attached to a distal end of the femur F. The femoral component 4 is formed of a metal material having biocompatibility (for example, a cobalt-chromium-molybdenum alloy) and a ceramic material (for example, a zirconia ceramic).

The femoral component 4 has a U-shape or a J-shape as viewed from the side when the femoral component 4 is installed on the femur F of the patient. The femoral component 4 covers an end portion of the femur F and also a front side and a rear side thereof. A slide portion 41 in contact with the tibial plate 5 described below, and a guide groove 42 provided in a front-and-rear direction are formed in the femoral component 4.

### Tibial Plate 5

The tibial plate 5 is used in combination with the tibial component 3 described below for repair or replacement of a tibial plateau of the knee joint. The tibial plate 5 is designed so as to form a joint with the femoral component 4. The tibial plate 5 may be fixed by fitting with a tibial tray 1 described below. The tibial plate 5 may be formed of a plastic material (for example, polyethylene) having biocompatibility and wear resistance. The tibial plate 5 is also called a “tibial insert”. A slide surface 51 on which the femoral component 4 slides, and a post portion 52 are formed on the tibial plate 5.

The post portion 52 protrudes upward, and is inserted into the guide groove 42 of the femoral component 4. When the patient flexes the knee, the post portion 52 of the tibial plate 5 moves back and forth along the guide groove 42.

### Tibial Component 3

The tibial component 3 is used for repair or replacement of the tibia T of the patient. The tibial component 3 is designed so as to form a joint with the femoral component 4. The tibial component 3 is attached to a proximal end (for example, the tibial plateau) of the tibia T. The tibial component 3 includes at least the tibial tray 1 (tray) and a tibial block 2 (augment member).

The tibial tray 1 includes an upper surface 12 in contact with the tibial plate 5, and a lower surface 11 (main surface) that is a surface opposite to the upper surface 12 and is a surface on a side in contact with the tibia T of the patient. The lower surface 11 of the tibial tray 1 may have a stem portion 15 protruding from the lower surface 11 and having a pillar or tubular shape, and a keel portion 14 protruding outward from a side surface of the stem portion 15.

The stem portion 15 includes a mechanism for connecting an extension stem 6 described below to the tibial tray 1. For example, when the stem portion 15 is a tubular member, a female thread that screws onto a male thread provided on the extension stem 6 may be formed in an inner wall of the stem portion 15.

The keel portion 14 is a protruding part provided such that the stem portion 15 inserted into a medullary cavity of the tibia T does not pivot inside the medullary cavity. In the present specification, the tibial tray 1 including two keel portions 14 is described as an example. However, the keel portion 14 is not an essential configuration for the tibial tray 1. Further, the number of the keel portions 14 is arbitrary and is not limited to two.

The tibial block 2 is an augment member used by being mounted on the lower surface 11 of the tibial tray 1. The tibial block 2 is optionally used for the purpose of increasing the supportability of the tibial component 3 by augmenting the lower surface 11 and a bone defect portion of the tibia T of the patient. The tibial block 2 may be disposed on at least a part on a right side with respect to the center of the lower surface 11 and on at least a part on a left side with respect to the center.

In the present disclosure, the tibial block 2 that covers a part (approximately half) of the lower surface 11 lower surface 11 is mainly described. However, the tibial block 2 is not limited thereto, and may have a size that covers equal to or greater than half or almost all of the lower surface 11.

Note that a tibial wedge (augment member) is a known augment member used for a purpose similar to that of the tibial block 2. The tibial block 2 has a semicircular plate shape having as substantially uniform thickness, whereas the tibial wedge has a wedge shape having a thinner thickness at one end portion than that at another end portion. In other words, an upper surface 22 (first surface) and a lower surface 21 (second surface) of the tibial block 2 are substantially parallel to each other, whereas an upper surface and a lower surface of the tibial wedge are not parallel to each other. The thickness of the tibial wedge is thinner from the right side to the left side or from the left side to the right side when the tibial wedge is installed on the tibia T of the patient, for example. Alternatively, the thickness is thinner from the rear side to the front side when the tibial wedge is installed on the tibia. In the present specification, the tibial component 3 in which the tibial block 2 is applied as the augment member is described as an example, but is not limited thereto. For example, the tibial wedge may be applied as an augment member instead of the tibial block 2 in the tibial component 3. Further, the tibial wedge may also have a size that covers equal to or greater than half or almost all of the lower surface 11.

The extension stem 6 is a member optionally used for the purpose of increasing the supportability of the tibial component. The extension stem 6 has a rod shape, and is inserted into the medullary cavity of the tibia T. Note that a protruding part 61 protruding outward from a surface of the extension stem may be formed such that the extension stem 6 does not pivot inside the medullary cavity. Note that the extension stem 6 is not an essential configuration for the tibial component 3 according to one aspect of the present invention. Therefore, FIG. 1 illustrates only a proximal portion of the extension stem 6, and does not illustrate a distal portion.

### Structure of Tibial Component 3

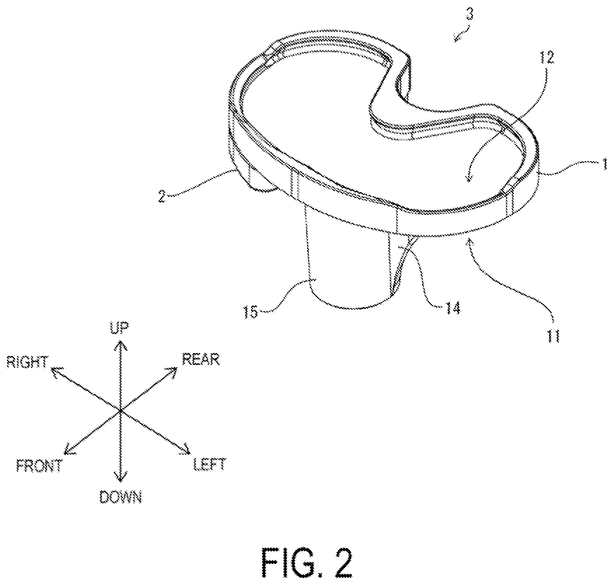

Next, a structure of the tibial component 3 will be described with reference to FIG. 2. FIG. 2 is a perspective view illustrating the tibial component 3. Note that, in the tibial component 3 illustrated in FIG. 2, the tibial block 2 is disposed on the tibial tray 1.

As illustrated in FIG. 2, a through hole extending from the lower surface 11 to the upper surface 12 is not formed in the tibial tray 1. Furthermore, a recessed portion is also not formed in the upper surface 12 of the tibial tray 1. By constituting the tibial tray 1 in this way, even when a contact surface of the tibial plate 5 and the upper surface 12 of the tibial tray 1 slide, the contact surface of the tibial plate 5 is not shaved. Note that an edge portion 121 fitting with the tibial plate 5 may be formed on an outer edge portion of the upper surface of the tibial tray 1.

### Structure of Tibial Tray 1

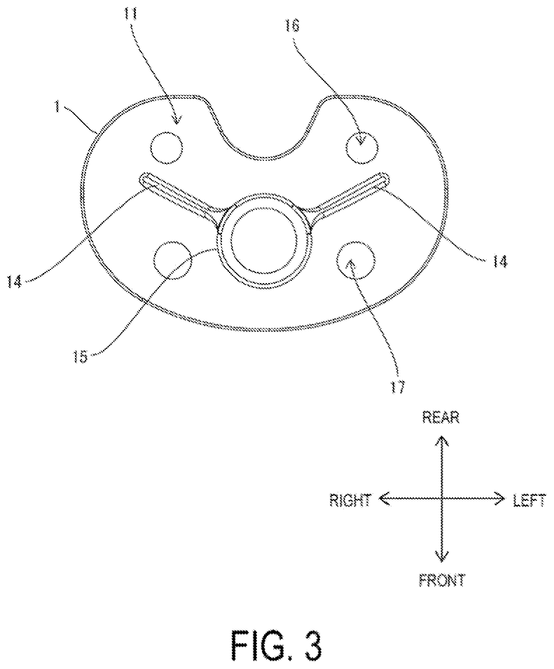

Next, a structure of the tibial tray 1 will be described with reference to FIG. 3. FIG. 3 is a plan view when the tibial tray 1 is viewed from below.

A recessed portion 16 and a recessed portion 17 that are both non-penetrating are formed in the lower surface 11 of the tibial tray 1 in addition to the keel portion 14 and the stem portion 15 described above. The recessed portion 17 is included in an engagement mechanism for engaging the tibial block 2 and the tibial tray 1. Note that the engagement mechanism will be described below with a specific example.

A position of a bone defect portion of the tibia T is different for each patient. Thus, the position to be augmented by the tibial block 2 may be located on the right side of the tibial tray 1, or may be located on the left side. Further, a bone defect may be large, and a portion from the right side to the left side of the lower surface 11 of the tibial tray 1, or an entire surface of the lower surface 11 may need to be augmented. Thus, as illustrated in FIG. 3, for example, a pair of the recessed portions 16 and a pair of the recessed portions 17 may be formed at left-right symmetrical positions in the lower surface 11 in the tibial tray 1. In the present embodiment, the lower surface 11 has a left-right symmetrical shape. As another embodiment of the tibial block, the tibial block 2 may have a size that covers equal to or greater than half of the lower surface 11, or may have a size substantially equal to the size of the lower surface 11 in plan view.

### Structure of Tibial Block 2

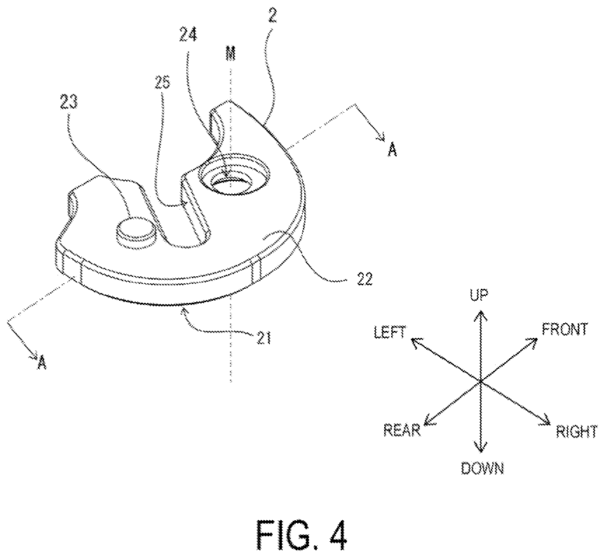

Subsequently, a structure of the tibial block 2 will be described with reference to FIGS. 4 to 7.

FIG. 4 is a perspective view illustrating the tibial block 2 except for a projecting portion 26. Although the projecting portion 26 is one component of the tibial block 2, FIG. 4 illustrates the tibial block 2 without the projecting portion 26 in order to illustrate the structure of a first through hole 24 described below.

A cut portion 25 is formed in the tibial block 2 illustrated in FIG. 4 such that the tibial block 2 can be mounted on the tibial tray 1 including the stem portion 15 and the keel portion 14. Specifically, the cut portion 25 is formed at a position corresponding to the position of the stem portion 15 and the keel portion 14 of the tibial tray 1 when the tibial block 2 is placed on the lower surface 11 of the tibial tray 1.

The tibial block 2 includes the upper surface 22 facing the lower surface 11 of the tibial tray 1, and the lower surface 21 that is a surface opposite to the upper surface 22. A cylindrical projecting portion 23 that can fit with the recessed portion 16 of the tibial tray 1 is formed on the upper surface 22 and the lower surface 21 of the tibial block 2. Further, the first through hole 24 in which the projecting portion 26 described below is mounted so as to be rotatable about an axis M (rotational axis) as a central axis is formed in the tibial block 2. Here, the axis M is intended to be the central axis of a cylindrical portion (body) of the projecting portion 26. The projecting portion 26 will be described below.

A narrow portion 240 protruding toward the inside of the first through hole 24 (that is, toward a side surface of the projecting portion 26) is formed on an inner wall of the first through hole 24. The size of a space surrounded by the narrow portion 240 of the first through hole 24 is narrower than the size of a space surrounded by the inner wall of the first through hole 24 near the upper surface 22 and the lower surface 21. In other words, in the first through hole 24, a portion in which the narrow portion 240 is formed is narrower than a size of an opening at an edge of the first through hole 24.

### Structure of Projecting Portion 26

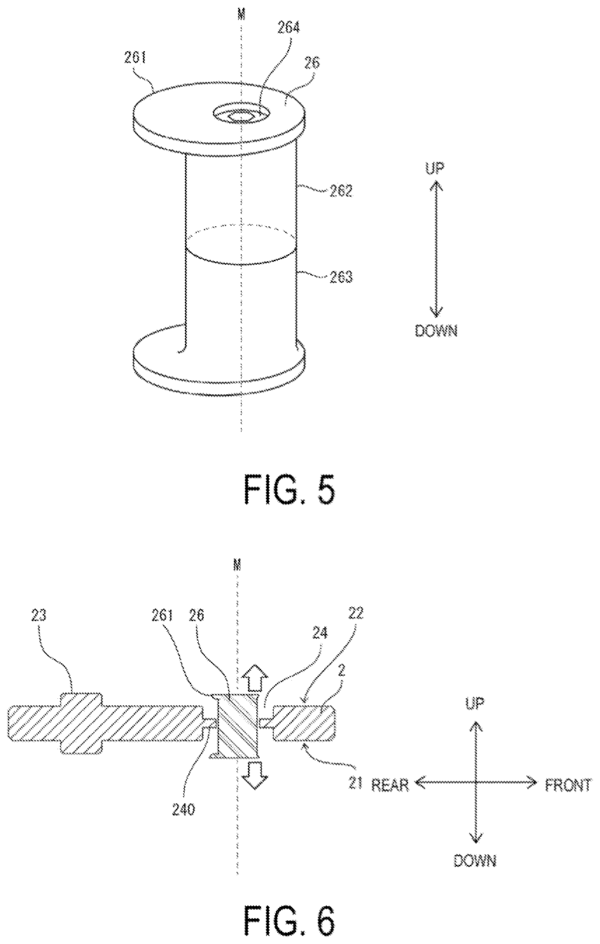

Here, a structure of the projecting portion 26 will be described with reference to FIG. 5. FIG. 5 is a perspective view illustrating the projecting portion 26 of the tibial block 2.

As illustrated in FIG. 5, the projecting portion 26 has a substantially cylindrical shape. The projecting portion 26 is disposed in the first through hole 24, and protrudes from the tibial block 2 toward the lower surface of the tibial tray 1.

An upper end and a lower end of the projecting portion 26 include a protruding part 261 formed to be eccentric with respect to the axis M. In other words, the axis M is configured not to pass through the center of the protruding part 261, and a distance from the axis M of the projecting portion 26 to an outer edge of the protruding part 261 is not uniform. In other words, a length in which the protruding part 261 protrudes in a predetermined direction toward the outside of the projecting portion 26 is different depending on the rotational angle of the projecting portion 26.

As illustrated in FIG. 5, a groove 264 having any shape corresponding to a tool (for example, a wrench) that can be used for rotating the projecting portion 26 may be formed in the upper end and the lower end of the projecting portion 26. In this way, a surgeon who performs surgery can rotate the projecting portion 26 by a desired rotational angle by using the tool. FIG. 5 illustrates the projecting portion 26 in which a hexagonal groove 264 is formed.

Note that the projecting portion 26 may have a configuration formed in combination with a plurality of portions. For example, a structure for fitting or screwing an upper projecting portion 262 and a lower projecting portion 263 may be formed on a lower end of the upper projecting portion 262 and an upper end of the lower projecting portion 263. In this case, the upper projecting portion 262 is inserted from the opening of the first through hole 24 in the upper surface 22, and the lower projecting portion 263 is inserted from the opening of the first through hole 24 in the lower surface 21, and the upper projecting portion 262 and the lower projecting portion 263 may be fitted (or screwed) so as to be integrated.

### Tibial Block 2 Including Projecting Portion 26

The tibial block 2 including the projecting portion 26 will be described with reference to FIG. 6. FIG. 6 is a cross-sectional view of the tibial block 2 including the projecting portion 26 taken along line A-A in FIG. 4. Note that FIG. 6 does not illustrate the cut portion 25 for simplicity.

In the present embodiment, the upper surface 22 of the tibial block 2 and the lower surface 21 of the tibial block 2 have a front-back symmetrical shape. In other words, in the tibial block 2 illustrated in FIG. 6, the projecting portion 23 is formed on not only the upper surface 22 but also on the lower surface 21. Further, the protruding part 261 is formed on the upper end and the lower end of the projecting portion 26. In this way, the tibial block 2 may be mounted on both of the right side and the left side of the tibial tray 1. Specifically, the tibial block 2 augments the bone defect portion on the left side in the tibia T of the patient when the upper surface 22 of the tibial block 2 is disposed so as to face the lower surface 11 of the tibial tray 1.

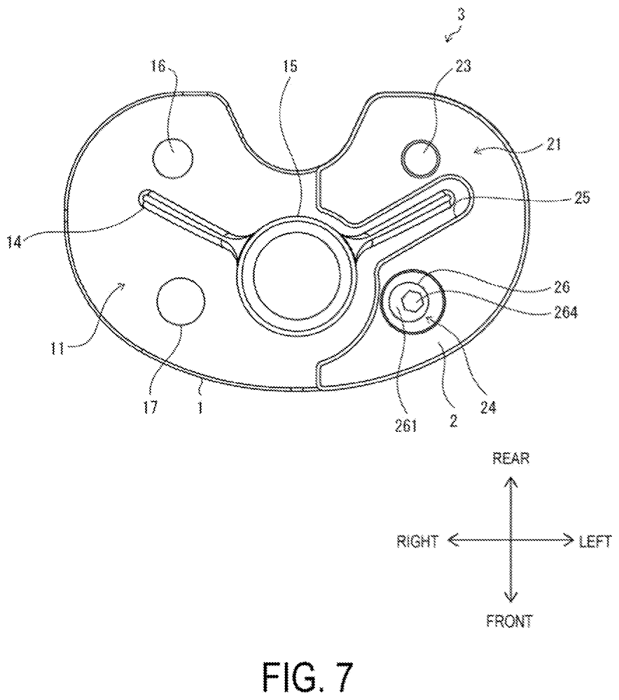

On the other hand, as illustrated in FIG. 7, the recessed portion 16 and the recessed portion 17 are also formed on the right side of the lower surface 11 of the tibial tray 1 similarly to the left side.

The tibial block 2 augments the bone defect portion on the right side in the tibia T of the patient when the lower surface 21 of the tibial block 2 is disposed so as to face the lower surface 11 of the tibial tray 1.

The protruding part 261 of the projecting portion 26 cannot pass through the space surrounded by the narrow portion 240 of the first through hole 24. Thus, the projecting portion 26 is not detached from the first through hole 24. The upper end of the projecting portion 26 can protrude toward the lower surface 11 of the tibial tray 1 until the protruding part 261 formed on the lower end of the projecting portion 26 comes into contact with the narrow portion 240 of the first through hole 24.

### Supplementary Note

Note that, as described above, the lower surface 11 of the tibial tray 1 may have a left-right symmetrical shape in plan view in the tibial component 3. When the tibial block 2 is disposed on the right side with respect to the center of the lower surface 11 or on the left side with respect to the center, the tibial component 3 for the left knee joint of the patient and the tibial component 3 for the right knee joint of the patient can be made common (that is, have a member having the same shape). Thus, the manufacturing cost of the tibial component 3 can be reduced.

Furthermore, the upper surface 22 and the lower surface 21 of the tibial block 2 may have a front-back symmetrical shape as described above. In this case, the tibial block 2 may be able to be fixed to the tibial tray 1 with the upper surface 22 in contact with a first side that is either the right side or the left side with respect to the center of the lower surface 11. Further, the tibial block 2 may be able to be fixed to the tibial tray 1 with the lower surface 21 in contact with a second side opposite to the first side. When this configuration is adopted, the tibial block 2 can be fixed to the tibial tray 1 by any of the upper surface 22 and the lower surface 21 of the tibial block 2 coming into contact with the right side or the left side with respect to the center of the lower surface 11 of the tibial tray 1. In this way, the same tibial block 2 having reversible sides can be mounted on both of the left side and the right side on the lower surface 11 of the tibial tray 1. Thus, the manufacturing cost of the tibial component 3 can be reduced.

### Engagement Mechanism and Fixing Mechanism

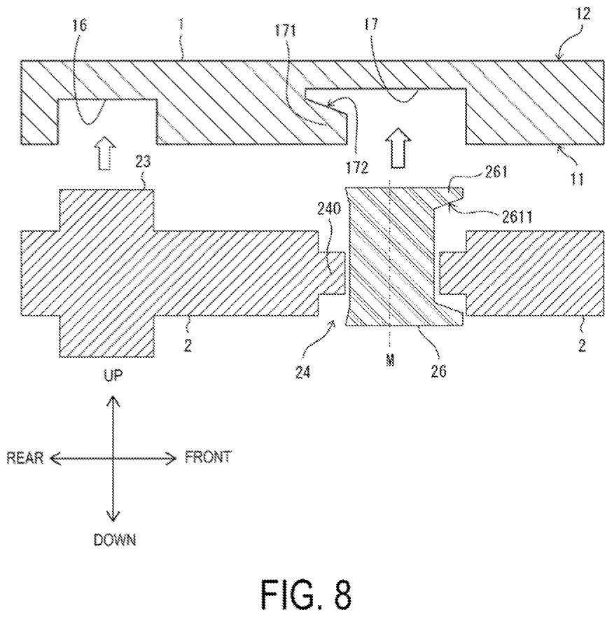

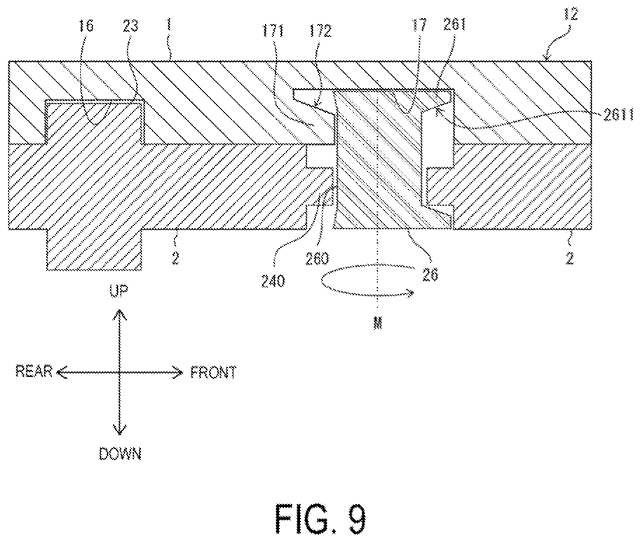

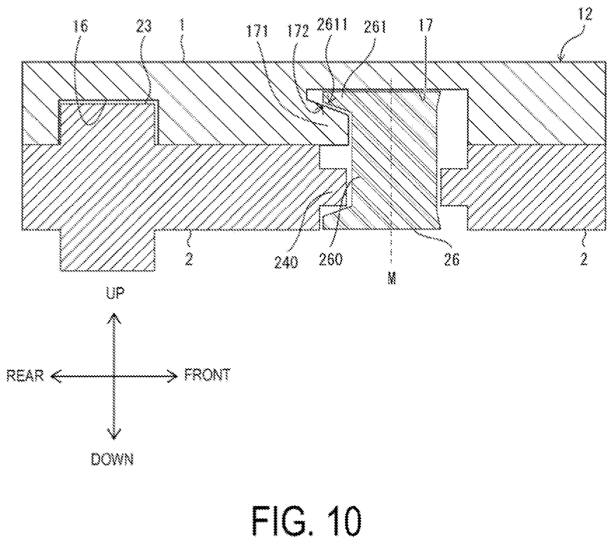

Next, the engagement mechanism configured to engage the tibial tray 1 and the tibial block 2, and the fixing mechanism for fixing the tibial block 2 to the tibial tray 1 will be described with reference to FIGS. 8 to 10. FIGS. 8 and 9 are schematic views for describing one example of the engagement mechanism configured to engage the tibial tray 1 and the tibial block 2. FIG. 10 is a schematic view illustrating one example of the fixing mechanism for fixing the tibial block 2 to the tibial tray 1.

The engagement mechanism includes the non-penetrating recessed portion 17 disposed in the lower surface 11 of the tibial tray 1, and the projecting portion 26 projecting from the tibial block 2 to the lower surface 11 side. The recessed portion 17 includes an overhanging portion 171 protruding more inward on an opening side than on a bottom portion side in at least a part of an inner wall. The projecting portion 26 includes the protruding part 261 that can engage with the overhanging portion 171. FIG. 8 illustrates a case where the overhanging portion 171 is formed on the inner wall on a rear side in the recessed portion 17; however, the overhanging portion 171 is not limited thereto.

The protruding part 261 includes a taper surface 2611 in which an upper end side (tip side) of the projecting portion 26 expands in a taper shape. The overhanging portion 171 includes an inclined surface 172 in which the inner wall is inclined along the taper surface 2611.

As illustrated in FIG. 8, the first through hole 24 of the tibial block 2 is disposed in a position facing the recessed portion 17 of the lower surface 11 of the tibial tray 1 when the tibial block 2 is placed in a mounting position on the lower surface 11 of the tibial tray 1.

As illustrated in FIG. 9, when the rotational angle of the projecting portion 26 is a release angle, the tibial block 2 is placed in the mounting position in the lower surface 11 of the tibial tray 1. Here, the “release angle” is intended to be an angle when the projecting portion 26 is rotated such that the protruding part 261 of the projecting portion 26 faces a side opposite to a position in which the overhanging portion 171 of the recessed portion 17 is formed. For example, the release angle in FIG. 9 is an angle at which the protruding part 261 of the projecting portion 26 greatly protrudes frontward. When the rotational angle of the projecting portion 26 is the release angle, the protruding part 261 can be inserted into the recessed portion 17 without being obstructed by the overhanging portion 171. The engagement mechanism is intended to be a mechanism for setting the rotational angle of the projecting portion 26 as the release angle, and engaging the projecting portion 26 with the recessed portion 17 without the protruding part 261 being obstructed by the overhanging portion 171.

After the projecting portion 26 is inserted into the recessed portion 17, as illustrated in FIG. 10, by rotating the rotational angle of the projecting portion 26 from the release angle to a mounting angle, the protruding part 261 and the overhanging portion 171 engage with each other. Here, the “mounting angle” is intended to be an angle when the projecting portion 26 is rotated such that the protruding part 261 of the projecting portion 26 faces the side on which the overhanging portion 171 of the recessed portion 17 is formed. When the rotational angle of the projecting portion 26 is the mounting angle, the taper surface 2611 of the protruding part 261 and the inclined surface 172 of the overhanging portion 171 engage with each other.

The fixing mechanism is intended to be a mechanism for suppressing rotation of the projecting portion 26 by friction between the overhanging portion 171 and the protruding part 261 due to contact between the overhanging portion 171 and the protruding part 261 when the rotational angle of the projecting portion 26 is the mounting angle. When such a fixing mechanism is adopted, the tibial block 2 can be mounted on the tibial tray 1 without using a part (for example, a positioning pin 29 and the like) other than the tibial tray 1 and the tibial block 2.

When such an engagement mechanism is adopted, the tibial block 2 can be mounted on the tibial tray 1 by a simple procedure without forming as through hole in the tibial tray 1.

As illustrated in FIGS. 8 to 10, the projecting portion 23 is disposed in the position facing the recessed portion 16 of the lower surface 11 of the tibial tray 1 when the tibial block 2 is placed in the mounting position in the lower surface 11 of the tibial tray 1. In the engagement mechanism and the fixing mechanism described above, fitting between the projecting portion 23 of the tibial block 2 and the recessed portion 16 of the tibial tray 1 may be further used.

As illustrated in FIG. 9, the projecting portion 26 is inserted into the recessed portion 17 and the projecting portion 23 is fitted into the recessed portion 16, and thus a state where the tibial block 2 is placed in the mounting position in the lower surface 11 of the tibial tray 1 can be maintained in a stable manner. Thus, a surgeon who performs surgery can easily perform the operation of rotating the projecting portion 26.

### Second Embodiment

Another embodiment of the present invention will be described below. Note that, for convenience of description, a member having the same function as that of a member described in the embodiment described above is denoted by the same reference sign, and description thereof will not be repeated.

### Structure of Tibial Tray 1a

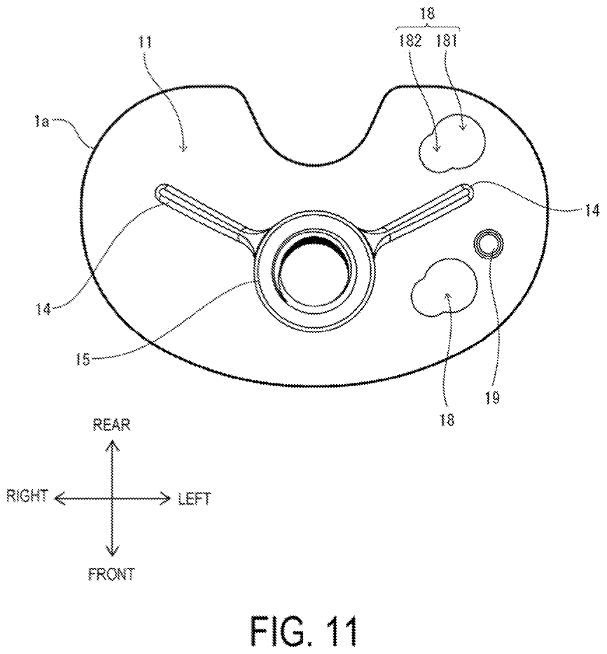

First, a structure of a tibial tray 1a will be described with reference to FIG. 11. FIG. 11 is a plan view when the tibial tray 1a is viewed from below.

A recessed portion 18 and a second recessed portion 19 (non-penetrating holes) that are both non-penetrating are formed in a lower surface 11 (main surface) of the tibial tray 1a in addition to a keel portion 14 and a stem portion 15. The recessed portion 18 is included in an engagement mechanism configured to engage a tibial block 2a described below and the tibial tray 1a. Meanwhile, the second recessed portion 19 is included in a fixing mechanism for fixing the tibial tray 1a to the tibial block 2a. The engagement mechanism and the fixing mechanism will be described below with a specific example.

The recessed portion 18 is a recessed portion formed of a first region 181 and a second region 182 in combination. Both of the first region 181 and the second region 182 are non-penetrating recessed portions formed in the lower surface 11 of the tibial tray 1a.

A projecting portion 27a protruding from an upper surface 22 of the tibial block 2a to the lower surface 11 side of the tibial tray 1a is inserted into the recessed portion 18. When the tibial block 2a horizontally moves in a front right direction on the lower surface of the tibial tray 1a, the projecting portion 27a inserted into the first region 181 is configured to be able to move toward the second region 182 due to the horizontal movement.

FIG. 11 illustrates the example in which two recessed portions 18 are formed, but the recessed portions 18 may be formed in a number that matches the number of the projecting portions 27a of the tibial block 2a. Further, FIG. 11 illustrates a case where the size of the recessed portion 18 formed in the front and the size of the recessed portion 18 provided in the rear are the same, but each of the recessed portions 18 may be formed in a size that matches the size of the projecting portion 27a inserted into each of the recessed portions 18.

The example illustrated in FIG. 11 illustrates the recessed portion 18 with an opening having a shape in which two circles having different radii overlap each other by shifting center positions. However, this is merely one example, and any shape (for example, a polygonal shape, an elliptical shape, and the like) that can realize the engagement mechanism described below may be applied as the shape (or the first region 181 and the second region 182) of the recessed portion 18.

Further, a case where the tibial block 2a is disposed on a left side of the tibial tray 1a is illustrated in FIG. 11, but is not limited thereto. The recessed portion 18 and the second recessed portion 19 may be formed on a right side in the lower surface 11 in the tibial tray 1a. Alternatively, the recessed portion 18 and the second recessed portion 19 may be formed at left-right symmetrical positions in the lower surface 11 in the tibial tray 1a.

### Structure of Tibial Block 2a

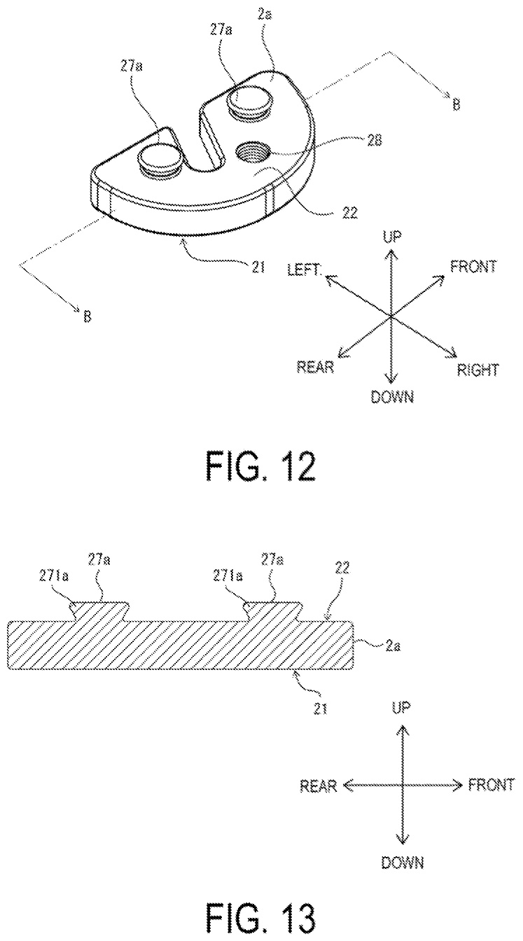

Subsequently, a structure of the tibial block 2a will be described with reference to FIGS. 12 and 13. FIG. 12 is a perspective view illustrating the tibial block 2a. FIG. 13 is a cross-sectional view of the tibial block 2a taken along line B-B in FIG. 12. Note that FIG. 12 does not illustrate a cut portion for avoiding the keel portion 14.

The tibial block 2a includes the upper surface 22 facing the lower surface 11 of the tibial tray 1a, and a lower surface 21 that is a surface opposite to the upper surface 22. As illustrated in FIG. 12, the projecting portion 27a that can be inserted into the recessed portion 18 of the tibial tray 1a is formed on the upper surface 22 of the tibial block 2a. Further, a second through hole 28 is formed in the tibial block 2a.

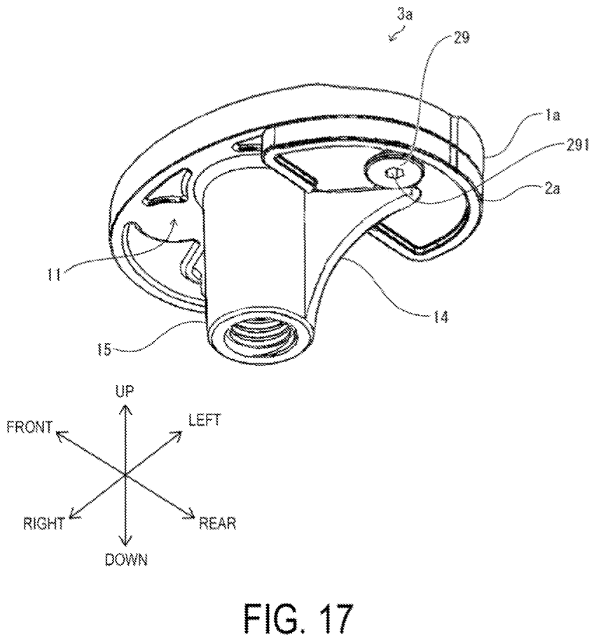

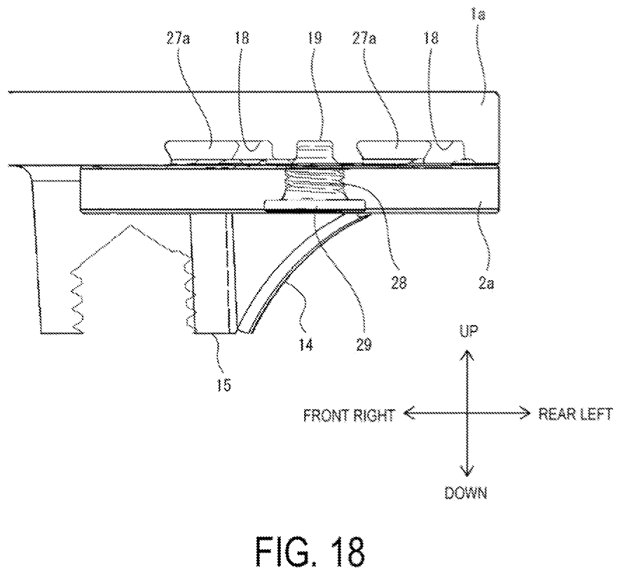

The second through hole 28 is, for example, a through hole into which a fixing member such as a positioning pin 29 illustrated in FIGS. 17 and 18 is inserted. The second through hole 28 is disposed in a position facing the second recessed portion 19 of the lower surface 11 of the tibial tray 1a.

A female thread may be formed in an inner periphery of the second through hole 28. In this case, the female thread formed in the inner periphery of the second through hole 28 and a male thread formed in at least a part of an outer periphery of the fixing member can be screwed together.

As illustrated, the number of second through holes 28 may be one, or may be more than one.

Two or more projecting portions 27a are preferably formed. When two or more projecting portions 27a are formed, a surgeon can easily insert the projecting portion 27a into the recessed portion 18 of the tibial tray 1a, and can also move the tibial block 2a from a release position to a mounting position in a stable manner.

As illustrated in FIG. 13, the projecting portion 27a includes a protruding part 271a. A tip of the projecting portion 27a is formed to be thicker than a base, and the tip is the protruding part 271.

### Engagement Mechanism

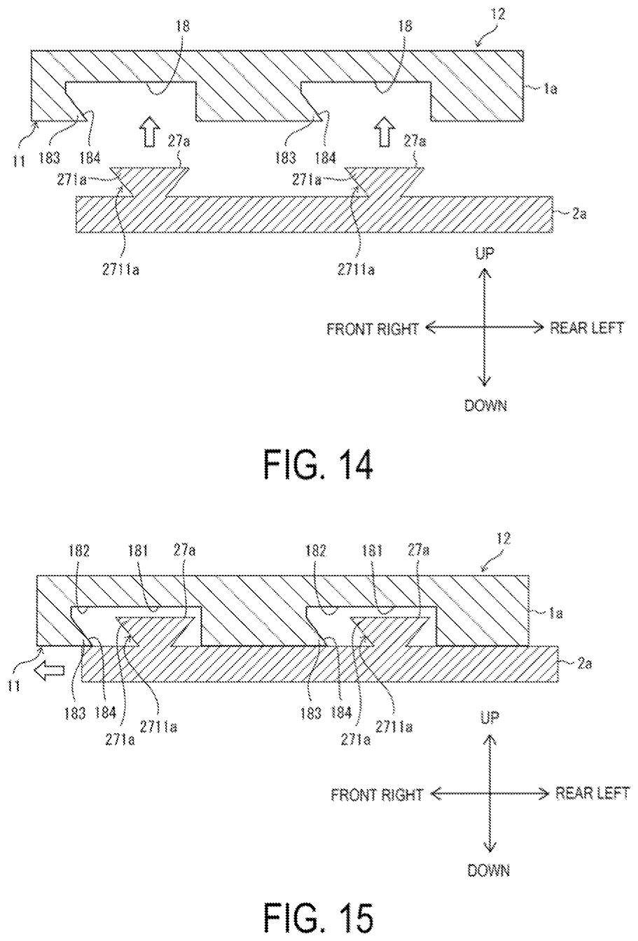

Next, the engagement mechanism for engaging the tibial tray 1a and the tibial block 2a will be described with reference to FIGS. 14 to 16. FIGS. 14 to 16 are schematic views for describing the engagement mechanism for engaging the tibial tray 1a and the tibial block 2a.

The engagement mechanism includes the non-penetrating recessed portion 18 disposed in the lower surface 11 of the tibial tray 1a, and the projecting portion 27a projecting from the tibial block 2a to the lower surface 11 side. The recessed portion 18 includes an overhanging portion 183 protruding more inward on an opening side than on a bottom portion side in at least a part of an inner wall. The projecting portion 27a includes the protruding part 271a that can engage with the overhanging portion 183. The overhanging portion 183 is not limited thereto, but FIG. 14 illustrates a case where the overhanging portion 183 is formed on the inner wall on a front right side (that is, corresponding to the second region 182) of the recessed portion 18.

The protruding part 271a includes a taper surface 2711a in which an upper end side (tip side) of the projecting portion 27a expands in a taper shape. The overhanging portion 183 includes an inclined surface 184 in which the inner wall is inclined along the taper surface 2711a.

As illustrated in FIG. 14, the projecting portion 27a is disposed at a position facing the recessed portion 18 of the lower surface 11 of the tibial tray 1a when the tibial block 2a is placed in the mounting position and the release position shifted from the mounting position in the lower surface 11 of the tibial tray 1a.

For example, as illustrated in FIG. 15, when the tibial block 2a is in the release position, the projecting portion 27a of the tibial block 2a may be inserted into the first region 181. In other words, by placing the tibial block 2a in the release position shifted from the mounting position in the lower surface 11 of the tibial tray 1a, the projecting portion 27a is inserted into the first region 181.

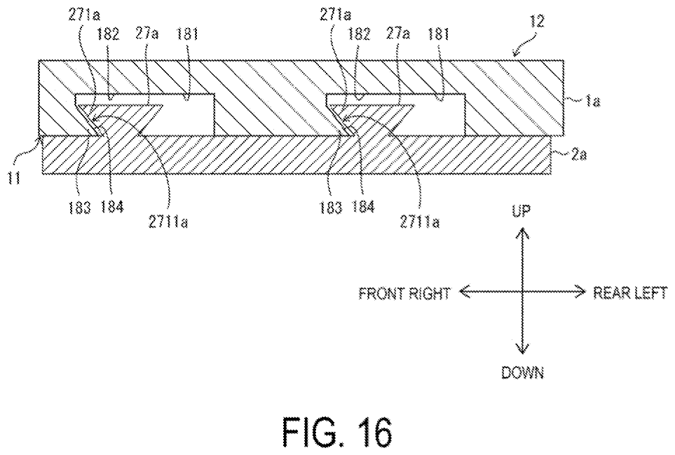

When the projecting portion 27a is inserted into the first region 181 and the tibial block 2a then moves to the mounting position, as illustrated in FIG. 16, the projecting portion 27a moves (slides) to the second region 182, and the protruding part 271a and the overhanging portion 183 engage with each other. In other words, by moving the tibial block 2a in a particular direction from the release position toward the mounting position, the protruding part 271a engages with the overhanging portion 183. In the example illustrated in FIGS. 15 and 16, the particular direction is a direction from a center position of the first region 181 toward a center position of the second region 182 (a direction from a rear left side toward a front right side).

When such an engagement mechanism is adopted, the tibial block 2a can be disposed in the mounting position of the tibial tray 1a by a simple procedure without forming a through hole in the tibial tray 1a.

### Fixing Mechanism

Subsequently, the fixing mechanism for fixing the tibial block 2a to the tibial tray 1a will be described with reference to FIGS. 17 and 18. FIG. 17 is a perspective view when a tibial component 3a in which the tibial block 2a is fixed to the tibial tray 1a by using the positioning pin 29 is viewed from below. FIG. 18 is a schematic view for describing the fixing mechanism for fixing the tibial tray 1a to the tibial block 2a.

The tibial block 2a placed in the mounting position in the lower surface 11 or the tibial tray 1a may be fixed by the fixing mechanism. As illustrated in FIG. 18, the fixing mechanism includes the second through hole 28 disposed in the tibial block 2a, the second recessed portion 19 being non-penetrating and being disposed in the lower surface 11 so as to face the second through hole 28, and a fixing member that can engage with the second recessed portion 19 via the second through hole 28.

The fixing member may be, for example, the positioning pin 29 (see FIG. 18) that is inserted from the second through hole 28, reaches the second recessed portion 19, and can engage with the second recessed portion 19 in a state where the projecting portion 27a engages with the recessed portion 18.

A male thread that can be screwed into a female thread formed in an inner periphery of the second through hole 28 is formed on at least a part of an outer periphery of the positioning pin 29. As illustrated in FIG. 17, a groove 291 having any shape corresponding to a tool (for example, a wrench) that can be used for rotating the positioning pin 29 is formed in a head portion of the positioning pin 29. In this way, a surgeon who performs surgery can rotate the positioning pin 29 inserted into the second through hole 28 by using the tool. FIG. 17 illustrates the positioning pin 29 in which the groove 291 having a hexagonal shape is formed.

### Modification Example

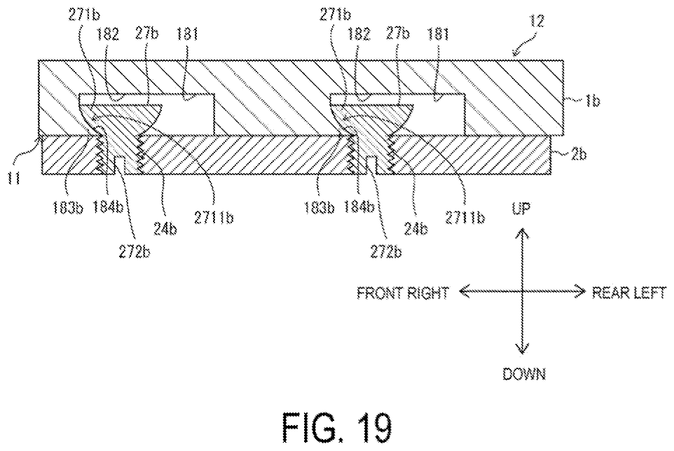

The tibial component 3a can be configured to fix a tibial block 2b to a tibial tray 1b without providing the second through hole 28, the positioning pin 29, and the second recessed portion 19. Such a configuration will be described with reference to FIG. 19. FIG. 19 is a schematic view for describing an engagement mechanism for engaging the tibial tray 1b and the tibial block 2b, and a fixing mechanism for fixing the tibial block 2b to the tibial tray 1b. Note that, for convenience of description, a member having the same function as that of a member described in the embodiments described above is denoted by the same reference sign, and description thereof will not be repeated.

As illustrated in FIG. 19, a male thread is formed in at least a part of an outer periphery of a projecting portion 27b. The male thread can be screwed into a female thread formed in a third through hole 24b formed in the tibial block 2b.

The projecting portion 27b is a screw member including a head portion 271b (protruding part) that is mounted on the third through hole 24b and has a dish shape or a trumpet shape. The bead portion 271b of the projecting portion 27b protrudes from an upper surface 22 of the tibial block 2b to a lower surface 11 side of the tibial tray 1b, and has a shape similar to that of the projecting portion 27a illustrated in FIGS. 13 to 16.

The head portion 271b can engage with an overhanging portion 183b. The head portion 271b includes a taper surface 2711b in which an upper end side (tip side) of the projecting portion 27b expands in a taper shape. Meanwhile, the overhanging portion 183 includes an inclined surface 184b in which an inner wall is inclined along the taper surface 2711b.

Meanwhile, a screw tip of the projecting portion 27b faces a lower surface 21 side of the tibial block 2b. As illustrated in FIG. 19, a groove 272b having any shape corresponding to a tool (for example, a wrench) that can be used for rotation the projecting portion 27b is formed in the screw tip of the projecting portion 27b. In this way, a surgeon who performs surgery can rotate the projecting portion 27b inserted into the third through hole 24b by using the tool from the lower surface 21 side.

For example, when the tibial block 2b moves from a release position to a mounting position, as illustrated in FIG. 19, the projecting portion 27b moves (slides) to a second region 182, and the head portion 271b and the overhanging portion 183b engage with each other. In other words, by moving the tibial block 2b in a particular direction from the release position toward the mounting position, the head portion 271b engages with the overhanging portion I83b.

The tibial block 2b placed in the mounting position in the lower surface 11 of the tibial tray 1b is fixed by the fixing mechanism. The fixing mechanism suppresses movement of the projecting portion 27b by friction between the overhanging portion 183b and the head portion 271b due to the projecting portion 27b being screwed into the tibial block 2b.

Specifically, when the projecting portion 27b is screwed into the tibial block 2b in a state where the head portion 271b of the projecting portion 27b engages with the overhanging portion 183b, the projecting portion 27b moves in a direction from up to down. The head portion 271b that moves from up to down pulls the overhanging portion 183b to the upper surface 22 of the tibial block 2b. In this way, the adhesion between the overhanging portion 183b and the head portion 271b is improved, and the projecting portion 27b returning to the release position is suppressed.

Here, the inclined surface 184b and the taper surface 2711b may be any curved surface that effectively causes friction between the overhanging portion 183b and the head portion 271b. For example, the larger the contact area between the inclined surface 184b and the taper surface 2711b, the larger the frictional force generated between the taper surface 2711b and the inclined surface 184b. Further, an inclination angle of the inclined surface 184b and the taper surface 2711b may be any angle that can tightly fix the tibial block 2b to the tibial tray 1b.

The present invention is not limited to each of the embodiments described above, and various modifications can be made within the scope indicated by the claims, and an embodiment obtained by appropriately combining technical means disclosed in different embodiments is also included in a technical scope of the present invention.

### Conclusion

<1> A tibial component according to one aspect of the present invention is a tibial component constituting an artificial knee joint, and includes: a tray including a main surface; an augment member disposed on the main surface of the tray; and an engagement mechanism configured to engage the augment member and the tray, wherein the engagement mechanism includes a recessed portion that is disposed in the main surface, is non-penetrating, and includes an overhanging portion protruding inward more on an opening side than on a bottom portion side in at least a part of an inner wall, and a projecting portion that protrudes from the augment member to the main surface side, and includes a protruding part configured to engage with the overhanging portion.

According to the configuration described above, the overhanging portion of the recessed portion that is disposed in the main surface of the tray and is non-penetrating engages with the protruding part of the projecting portion protruding from the augment member to the main surface side of the tray, and thus the augment member is mounted on the main surface side of the tray. In this way, the augment member can be mounted on the tray by a simple procedure without forming a through hole in the tray.

<2> In the tibial component, the projecting portion may be rotatably mounted on the augment member, and may also include a protruding part formed to be eccentric with respect to a rotational axis, by placing the augment member in a mounting position on the main surface of the tray when a rotational angle of the projecting portion is a release angle, the projecting portion may be inserted into the recessed portion, and, by rotating the rotational angle of the projecting portion from the release angle to a mounting angle, the protruding part may engage with the overhanging portion.

According to the configuration described above, by rotating the projecting portion with respect to the augment member, mounting/releasing of the augment member with respect to the tray can be switched.

<3> The tibial component may further include a fixing mechanism for fixing the augment member and the tray, and the fixing mechanism may suppress rotation of the projecting portion by friction between the overhanging portion and the protruding part due to contact between the overhanging portion and the protruding part when the rotational angle of the projecting portion is the mounting angle.

<4> In the tibial component, the main surface may have a left-right symmetrical shape in plan view, and the augment member may be disposed on a right side with respect to the center of the main surface or on a left side with respect to the center.

According to the configuration described above, the tibial component for a knee joint of a right leg of a patient and the tibial component for a knee joint of a left leg of the patient can be made into a common member, and the manufacturing cost of the tibial component 3 can be reduced.

<5> In the tibial component, the augment member may include a first surface and a second surface opposite to the first surface, and the first surface and the second surface may have a front-back symmetrical shape.

<6> In the tibial component, the augment member may be configured to be fixed to the tray with the first surface in contact with a first side that is either the right side or the let side with respect to the center of the main surface, and the augment member may be configured to be fixed to the tray with the second surface in contact with a second side opposite to the first side.

According to the configuration described above, the same augment member having reversible sides can be mounted on both of the left side and the right side of the main surface, and a manufacturing cost of a member (for example, the augment member) of the tibial component 3 can be suppressed.

<7> The augment member of the tibial component may be disposed on at least a part on a right side with respect to the center of the main surface and on at least a part on a left side with respect to the center.

According to the configuration described above, even when a bone defect portion of a tibia of a patient is present on either the right side or the left side, the augment member can be adapted to the bone defect portion. Note that, in this configuration, the augment member may have a size that covers almost all of the main surface.

<8> In the tibial component, by placing the augment member in a release position shifted from a mounting position in the main surface, the projecting portion may be inserted into the recessed portion, and, by moving the augment member in a particular direction from the release position toward the mounting position, the protruding part may engage with the overhanging portion.

According to the configuration described above, by moving the augment member in the particular direction described above, mounting/releasing of the augment member with respect to the tray can be switched.

<9> The tibial component may further include a fixing mechanism for fixing the augment member and the tray, and the fixing mechanism may include a through hole disposed in the augment member, a second recessed portion being non-penetrating and being disposed in the main surface to face the through hole, and a fixing member configured to engage with the second recessed portion via the through hole.

According to the configuration described above, the augment member mounted on the tray by the engagement mechanism is fixed by the fixing member configured to engage with the second recessed portion of the tray via the through hole disposed in the augment member. In this way, the augment member can be fixed by a simple procedure without forming a through hole in the tray.

<10> In the tibial component, the fixing mechanism may include a non-penetrating hole formed in the main surface, a through hole formed in the augment member, and a positioning pin configured to be inserted from the through hole and reach the non-penetrating hole in a state where the projecting portion engages with the recessed portion.

<11> In the tibial component, a female thread may be formed in an inner periphery of the through hole, and a male thread configured to be screwed into the female thread may be formed in at least a part of an outer periphery of the positioning pin.

<12> The tibial component may further include a fixing mechanism for fixing the augment member and the tray, by screwing a male thread formed in an outer periphery of the projecting portion into a female thread formed in the augment member, the projecting portion may be mounted on the augment member, and the fixing mechanism may suppress movement of the projecting portion by friction between the overhanging portion and the protruding part due to the projecting portion being screwed into the augment member.

<13> In the tibial component, the protruding part may include a taper surface in which a tip side of the projecting portion expands in a taper shape, and the overhanging portion may include an inclined surface in which the inner wall is inclined along the taper surface.

According to the configuration described above, the engagement mechanism (the engagement mechanism such as a “dovetail” used in a construction field) is obtained by the taper surface coming into contact with the inclined surface. Thus, as compared with a configuration including a recessed portion having a T-groove shape, required machining accuracy is less stringent. Further, as compared with a configuration having a recessed portion as a non-penetrating screw hole, a depth of a hole can be more easily set to be shallow, and a tray can be more easily set to be thin.

<14> In the tibial component, the tray may include a stem portion protruding from the main surface, and the augment member may include a cut portion configured to avoid the stem portion when the augment member is fixed to the main surface.

<15> In the tibial component, the tray may include a keel portion, the keel portion may protrude from the stem portion and/or the main surface, and the augment member may include the cut portion configured to avoid the stem portion and the keel portion when the augment member is fixed to the main surface.

According to the configuration described above, the augment member can also be applied to the tray including the stem portion and the keel portion.

### REFERENCE SIGNS LIST

1, 1a, 1b Tibial tray (tray)
2, 2a, 2b Tibial block (augment member)
3, 3a Tibial component
11 Lower surface (main surface)
12 Upper surface
17, 18 Recessed portion
19 Second recessed portion (non-penetrating hole)
21 Lower surface (second surface)
22 Upper surface (first surface)
26, 27a, 27b Projecting portion
28 Second through hole (through hole)
29 Positioning pin (fixing member)
171, 183, 183b Overhanging portion
172, 184, 184b Inclined surface
261, 271a Protruding part
271b Head portion (protruding part)
2611, 2611a, 2711b Taper surface

1. A tibial component constituting an artificial knee joint, comprising:
a tray comprising a main surface;
an augment member disposed on the main surface of the tray; and
an engagement mechanism configured to engage the augment member and the tray, wherein
the engagement mechanism comprises
a recessed portion that is disposed in the main surface, is non-penetrating, and comprises an overhanging portion protruding inward more on an opening side than on a bottom portion side in at least a part of an inner wall, and
a projecting portion that protrudes from the augment member to the main surface side, and comprises a protruding part configured to engage with the overhanging portion.

2. The tibial component according to claim 1, wherein
the projecting portion is rotatable mounted on the augment member, and also comprises a protruding part formed to be eccentric with respect to a rotational axis,
the projecting portion is inserted into the recessed portion by placing the augment member in a mounting position in the main surface of the tray when a rotational angle of the projecting portion is a release angle, and
the protruding part engages with the overhanging portion by rotating the rotational angle of the projecting portion from the release angle to a mounting angle.

3. The tibial component according to claim 2, further comprising
a fixing mechanism configured to fix the augment member and the tray, wherein
the fixing mechanism suppresses rotation of the projecting portion by friction between the overhanging portion and the protruding part due to contact between the overhanging portion and the protruding part when the rotational angle of the projecting portion is the mounting angle.

4. The tibial component according to claim 1, wherein
the main surface has a left-right symmetrical shape in plan view, and the augment member is disposed on a right side with respect to the center of the main surface or on a left side with respect to the center.

5. The tibial component according to claim 4, wherein
the augment member comprises a first surface and a second surface opposite to the first surface, and the first surface and the second surface have a front-back symmetrical shape.

6. The tibial component according to claim 5, wherein
the augment member is configured to be fixed to the tray with the first surface in contact with a first side that is either the right side or the left side with respect to the center of the main surface, and the augment member is configured to be fixed to the tray with the second surface in contact with a second side opposite to the first side.

7. The tibial component according to to claim 1, comprising
the augment member disposed on at least a part on a right side with respect to the center of the main surface and on at least a part on a left side with respect to the center.

8. The tibial component according to claim 1, wherein,
by placing the augment member in a release position shifted from a mounting position in the main surface, the projecting portion is inserted into the recessed portion, and
by moving the augment member in a particular direction from the release position toward the mounting position, the protruding part engages with the overhanging portion.

9. The tibial component according to claim 8, further comprising
a fixing mechanism configured to fix the augment member and the tray, wherein
the fixing mechanism comprises
a through hole disposed in the augment member,
a second recessed portion being non-penetrating and being disposed in the main surface to face the through hole, and
a fixing member configured to engage with the second recessed portion via the through hole.

10. The tibial component according to claim 9, wherein
the fixing mechanism comprises a non-penetrating hole formed in the main surface, a through hole formed in the augment member, and a positioning pin configured to be inserted from the through hole and reach the non-penetrating hole in a state where the projecting portion engages with the recessed portion.

11. The tibial component according to claim 10, wherein
a female thread is formed in an inner periphery of the through hole, and a male thread configured to be screwed into the female thread is formed in at least a part of an outer periphery of the positioning pin.

12. The tibial component according to claim 8, further comprising
a fixing mechanism for fixing the augment member and the tray, wherein,
by screwing a male thread formed in an outer periphery of the projecting portion into a female thread formed in the augment member, the projecting portion is mounted on the augment member, and
the fixing mechanism suppresses movement of the projecting portion by friction between the overhanging portion and the protruding part due to the projecting portion being screwed into the augment member.

13. The tibial component according to claim 1, wherein
the protruding part comprises a taper surface in which a tip side of the projecting portion expands in a taper shape, and
the overhanging portion comprises an inclined surface in which the inner wall is inclined along the taper surface.

14. The tibial component according to claim 1, wherein
the tray comprises a stem portion protruding from the main surface, and the augment member comprises a cut portion configured to avoid the stem portion when the augment member is fixed to the main surface.

15. The tibial component according to claim 14, wherein
the tray comprises a keel portion, the keel portion protrudes from the stem portion and/or the main surface, and the augment member comprises the cut portion configured to avoid the stem portion and the keel portion when the augment member is fixed to the main surface.
